(19) United States
(12) Patent Application Publication
(10) Pub. No.: US 2015/0025027 A1
(43) Pub. Date: Jan. 22, 2015

(54) Combination of a Monosubstituted Sulfamate Derivate of the Natural Monosaccharide d-Fructose (Topiramate) with an Anti-Depressant from the Phenyl Ketone Class (Bupropion) for Treating Obesity and Plurimetabolic Syndromes

(71) Applicants: PHARTRIALS PERQUISAS FARMACÊUTICAS LTDA, Goiânia - GO (BR); VENCIO & STIVAL MEDICOS ASSOCIADOS S/S LTDA (EPP), Goiânia - GO (BR)

(72) Inventors: Alessandro Rios Stival Moreira, Serrinha. Goiania - GO (BR); Cleverson Tinoco Feltrin, Goiania - GO (BR); Leonardo De Souza Teixeira, Goiania - GO (BR); Sergio Alberto Cunha Vencio, Goiania - GO (BR)

(21) Appl. No.: 14/383,430

(22) PCT Filed: Mar. 1, 2013
(86) PCT No.: PCT/BR2013/000058

§ 371 Date: Sep. 5, 2014

(30) Foreign Application Priority Data
Mar. 5, 2012 (BR) 10 2012 004888 4

(51) Int. Cl. A61K 31/7048; A61K 31/135

(52) U.S. Cl. CPC A61K 31/7048; A61K 31/135
USPC 514/27

(57) ABSTRACT

Combination of a monosubstituted sulfamate derivate of the natural monosaccharide d-Fructose (Topiramate) with an anti-depressant from the phenyl ketone class (Bupropion) for treating obesity and plurimetabolic syndromes, which takes into consideration “Combined Therapy”, using two drugs (topiramate and bupropion) with different action mechanisms, with the intent of promoting a synergistic effect on weight loss. The solid pharmaceutical form contains from 75 to 400 mg of bupropion chlorohydrate combined with 25 to 200 mg of topiramate; the presential are added in the formulation until q.s.p 1 pill/coated pill.

### BRIEF PRESENTATION

This patent of invention application is a Combination of a monosubstituted sulfamate derivate of the natural monosaccharide d-Fructose (Topiramate) with an anti-depressant from the phenyl ketone class (Bupropion) for treating obesity and plurimetabolic syndromes, which takes into consideration “Combined Therapy”, using two drugs (topiramate and bupropion) with different action mechanisms, with the intent of promoting a synergistic effect on reducing body weight.

Body weight is homeostatically regulated to preserve an individual's current weight. When body weight deviates from this level, diverse regulatory mechanisms are activated to restore the weight to previous levels. Thus, establishing a given molecular pathway as a target may lead to weight loss, however compensatory homeostatic responses will be activated, minimizing the efficacy of the drug. In an analogy with the treatment of other diseases, such as systemic high blood pressure (which uses the association of an ACE inhibitor with a thiazide) and type 2 diabetes (which uses the combination of metformin and sulfonylurea), two substances would be used with different action mechanisms, in an attempt to maximize the desired effect and, simultaneously, diminish the incidence of adverse effects.

### FIELD OF APPLICATION

Treatment of obesity and plurimetabolic diseases.

### Techniques Currently Considered Efficient

New drugs and drug combinations with different proposals and action mechanisms are needed for treating the obese population.

There is a current perception by many specialists that the best way to develop safe and effective treatment for obesity would be through a combination of two drugs that could control appetite and promote satiety. It is also known that anxiety and compulsion are closely related in the etiology of excessive weight gain or in the difficulty in losing or maintaining body weight.

### BACKGROUND OF THE INVENTION

The growth in obesity over recent decades in Brazil and in the world is alarming. The exponential increase in cases of patients undergoing bariatric surgery is also clear, reflecting an effort by doctors to try to revert potential complications associated with the disease and, at the same time, expose the small range of pharmacological options for clinical treatment.

At present, in Brazil, there are only two drug options available for treating obesity approved by ANVISA. They are sibutramine and orlistat. Orlistat is a drug with little efficacy in weight loss. Its advantage is that it is exempt from adverse effects in the cardiovascular system and it can be used safely among the obese population. Furthermore, some studies demonstrate that this drug can also prevent diabetes (Xendos Study). Sibutramine is the most studied drug until today and also one of the most prescribed, with good results and a favorable profile in terms of adverse events. However, recently published data from the SCOUT (Sibutramine Cardiovascular OUTcomes Trial) study, which aimed to assess whether the medication was capable of reducing cardiovascular events (including AMI, CVA, cardiorespiratory failure or death) in a high-risk, obese population with type 2 diabetes (DM2), patients with a prior history of cardiovascular events and patients with another risk factor and/or history of prior cardiovascular events. There was a slight, although significant increase in these outcomes in the group of patients receiving sibutramine (11.4% vs. 10%), motivating the EMA (European Medicines Agency) and the FDA (Food and Drug Administration) to suspend sales of the drug in Europe and the USA. ANVISA opted not to suspend sibutramine sales, but it issued an opinion restricting use of the drug and prohibiting the use of amfepramone, fenproporex and mazindol due to the cardiovascular risk stemming from the noradrenergic effects of these drugs.

### STATE OF THE TECHNIQUE

The use of drugs for weight loss is effective in the short term; however, after one year of treatment, the currently most used drugs approved by the regulatory bodies (sibutramine and orlistat) generate placebo-subtracted weight loss of 3 to 5 kg on average. Therefore, it can be affirmed that both present slight efficacy, especially if compared to the weight loss achieved by patients who undergo bariatric surgery (approximately 20 to 25% of total weight with gastric bypass), resulting in a significant reduction in diseases associated with obesity and an increase in life expectancy. It thus becomes necessary to develop new drugs with greater efficacy, associated with a favorable safety profile and with minimal adverse events.

The pharmacological treatment of obesity has undergone a turbulent moment, and in Brazil, the sale of three drugs that had been the pillars of chemical treatment of obesity for decades was prohibited, since there was no admissible profile of safety, leaving millions of Brazilian without an acceptable variety of therapeutic options. It thus becomes imperative to intensify the studies of new drugs and their associations with different proposals and action mechanisms, aimed at offering appropriate treatment to the growing population of obese persons.

### OF THE INVENTION

The pro-opiomelacortin (POMC) in the neurons of the hypothalamic nucleus (also called the infundibulum) integrates the central and peripheral signals related to the balance of energy in the body and produces a network of anorexigenic compounds, where the main compound is the leptin hormone. Obese patients suffer resistance to leptin due to the decrease in POMC basal activity. Thus, agents that stimulate POMC are of interest in the pharmacotherapy treatment of obesity. Studies with POMC agonist agents demonstrate that they have adverse events and monotherapy reveals a modest reduction in body weight. Thus, combined therapies with drugs with complementary or synergistic effects are of global interest for obesity treatment.

The association of the two already existing medications (bupropion and topiramate) may be a faster and less costly solution, placing in the market a new medication capable of meeting the expectations of doctors and of millions of patients who suffer daily from excess weight and all the resulting complications.

Bupropion is an aminoketone with an action mechanism that slightly inhibits the recapturing of norepinefrine, serotonin and dopamine, which promoted weight loss in several studies, with a low incidence of adverse effects. This drug also has moderate anticholinergic effects.

Topiramate is an analogous substituted sulfamate of fructose 1.6-diphosphate that induces weight loss, where studies on humans suggest that it plays a role in reducing calorie ingestion, in the hormonal involvement of obesity and in the metabolism of glucose and lipids, while also having a low incidence of adverse effects.

The combination of these two drugs permits a synergistic effect, since each of them acts on a given physiological system for weight loss in overweight or obese patients, enabling a reduction in the dosages for each and thus reducing possible adverse events stemming from treatment with this combination.

### DETAILED DESCRIPTION OF THE INVENTION

Combination of bupropion chlorohydrate and topiramate.

Bupropion chlorohydrate is a monocyclic aminoketone unrelated to tricyclic antidepressants, with a molecular weight of 239.74 g. Bupropion chlorohydrate is an agent approved by the FDA for treating depression and for helping patients who need to quit smoking, and studies conducted by several authors (Jain et al, 2002; Anderson et al, 2002; Gadde et al, 2001) demonstrated it is effective in weight loss in humans. After oral administration, this drug is extensively metabolized by hydroxylation and reduction forming hydroxybupropion as its main metabolite and, in a smaller scale the metabolites erythrobupropion and threohydrobupropion. Thus, the hepatic clearance is the main route for eliminating this drug, and only 0.5% of an oral dose is excreted unaltered in the urine. Bupropion binds to 84% of the plasma proteins and its half-life is 24 hours.

Bupropion is chemically known as 1-(3-chlorophenyl)-2-{(1,1-dimethyl)-amino]-1-propanone, with a molecular weight of 239.74 g. Its molecular form is C₁₃H₁₈ClNo, with a pa of 7.9, solubility in water of 312 mg/ml and log P of 3.6, as per the structural formula below:

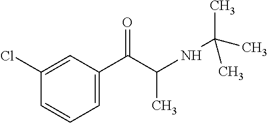 [SMILES: CC(NC(C)(C)C)C(=O)c1cccc(Cl)c1]

Topiramate is a fructose derivative approved by the FDA for treating epilepsy. It has multiple action mechanisms, blocking the action of calcium channels, inhibiting glutamate receptors, increasing the opening of chloride channels mediated by GABA, inhibiting carbon anhydrase and increasing potassium conductance. Studies conducted by several authors (Bays, 2004; Verrotti et al, 2011; Eliasson et al, 2007; Zilberstein et al, 2004; Rosenstock et al, 2007) demonstrated that his drug is also effective in weight loss in humans. The potential mechanism through which topiramate induces weight loss suggested in animal studies includes reduced energy efficiency, hypothalamic involvement, neurovascular and sensitivity to insulin, whereas studies in humans suggest a role in the ingestion of fewer calories, hormonal involvement and alterations in the metabolism of glucose and lipids. This drug has oral bioavailability of 80% and its peak plasma concentration is reached 2 hours after oral administration. It binds with plasma proteins at 15 to 41% and it is not extensively metabolized, with 70% of the dose administered eliminated through the urine. The other 30% of the dose is metabolized, forming six metabolites through hydroxylation, hydrolysis and glucuronidation reactions, with none corresponding to more than 5% of the dose administered. The half-life of this drug is between 19 and 23 hours. Plasma concentrations in the state of equilibrium are achieved after 4 days of treatment.

Topiramate is chemically known as 2,3,4,5-bis-O-(1-methylene)-β-D-fructopyranose sulfamate, with a molecular weight of 339.36 g. Its molecular form is C₁₂H₂₁NO₈S, with a log P of −0.7 and solubility in water of 9.8 mg/ml, as per the structural formula below:

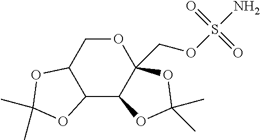 [SMILES: CC1(C)OC2CO[C@@]3(COS(N)(=O)=O)OC(C)(C)O[C@H]3C2O1]

The combination of bupropion chlorohydrate+topiramate can be in solid pharmaceutical form, containing some presential such as micro crystalline cellulose, hydroxypropyl methyl cellulose, hydroxylate, cysteine chlorohydrate, magnesium stearate, silica dioxide, polyethylene glycol, titanium dioxide, polysorbate 80, macrogol, monohydrated lactose, glycolysis sodium amide, cellulose acetate, povidone, lauryl sodium sulfate, sucrose, dyes and carnauba wax q.s.p. 1 pill/coated pill

Bupropion and topiramate are drugs that act on the central nervous system, where bupropion is considered an antidepressant agent and topiramate an anticonvulsant agent, but both are effective in weight loss, separately, as described by several authors (Jain et al, 2002; Anderson et al, 2002; Gadde et al, 2001; Bays, 2004; Verrotti et al, 2011; Eliasson et al, 2007; Zilberstein et al, 2004; Rosenstock et al, 2007).

The objective of the granulation is to transform post-crystalline or amorphous particles into solid aggregates of varied resistance and porosity. The granulate has some advantages: better conservation of component distribution homogeneity, greater density, greater ease of flow, greater compressibility and higher mechanical resistance. The ideal granulate should have homogeneous shape and color, low degree of granulometric distribution (less than 10% of the free primary particles or agglomerates of low granulometry), good fluidity, sufficient mechanical resistance and given degree of moisture.

Thus, the bupropion chlorohydrate+topiramate granulate will be produced by wet granulation (using an organic solvent or water to promote particle adherence), for 0.4 to 4 hrs., at a temperature of 20 to 70° C., or dry granulation (where pressure is responsible for cohesion of primary particles, with primary adjuvants called agglutinants such as micro crystalline cellulose, lactose, sucrose and others can be used), for 0.5 to 6 hrs., at a pressure of 0.5 to 10 Bar, at a temperature of 20 to 70° C. or by fluidized bed (where the particles of a drug or adjuvant are suspended under an ascending air current, receive a spray of dispersion or granulation solvent, resulting in the formation of granulates or pellets) or using the spray-drying technique, which promotes quick drying of solutions, suspensions and/or pasty substances, obtaining a dry and pulverized granulate.

After the granulation process, the mixture containing bupropion chlorohydrate+topiramate will undergo a compression process until obtaining pills/coated pills with adequate hardness and friability, disintegration and dissolution.

Solid, pharmaceutical form containing 75 to 400 mg of bupropion chlorohydrate combined with 25 to 200 mg of topiramate. The presential were added in the formulation until q.s.p 1 pill/coated pill.

1. Combination of a monosubstituted sulfamate derivate of the natural monosaccharide d-Fructose (Topiramate) with an anti-depressant from the phenyl ketone class (Bupropion) for treating obesity and plurimetabolic syndromes, which applies to the combination of two already existing drugs (bupropion and topiramate), a quick and less costly solution wherein there is a specific bupropion chlorohydrate+topiramate granulate for controlling obesity and plurimetabolic syndromes, through therapies combined with two drugs with complementary and synergistic effects for treating obesity.

2. Combination of a monosubstituted sulfamate derivate of the natural monosaccharide d-Fructose (Topiramate) with an anti-depressant from the phenyl ketone class (Bupropion) for treating obesity and plurimetabolic syndromes, in accordance with claim 1, wherein the combination of the bupropion chlorohydrate+topiramate drugs is presented in a solid pharmaceutical form.

3. Combination of a monosubstituted sulfamate derivate of the natural monosaccharide d-Fructose (Topiramate) with an anti-depressant from the phenyl ketone class (Bupropion) for treating obesity and plurimetabolic syndromes, in accordance with claims 1 and 2, wherein the solid pharmaceutical form contains 75 to 400 mg of bupropion chlorohydrate combined with 25 to 200 mg of topiramate; the presential are added in the formulation until q.s.p 1 pill/coated pill.

4. Combination of a monosubstituted sulfamate derivate of the natural monosaccharide d-Fructose (Topiramate) with an anti-depressant from the phenyl ketone class (Bupropion) for treating obesity and plurimetabolic syndromes, in accordance with claims 1 or 3, wherein the bupropion chlorohydrate+topiramate granulate is produced by wet granulation or by dry granulation, by fluidized bed, by the spray-drying technique.

5. Combination of a monosubstituted sulfamate derivate of the natural monosaccharide d-Fructose (Topiramate) with an anti-depressant from the phenyl ketone class (Bupropion) for treating obesity and plurimetabolic syndromes, in accordance with claims 1 or 4, wherein the mixture contains bupropion chlorohydrate+topiramate going through a direct or indirect compression process until obtaining pills/coated pills with adequate hardness and friability, disintegration and dissolution.

6. Combination of a monosubstituted sulfamate derivate of the natural monosaccharide d-Fructose (Topiramate) with an anti-depressant from the phenyl ketone class (Bupropion) for treating obesity and plurimetabolic syndromes, in accordance with claims 1 or 5, wherein wet granulation is conducted in 0.5 to 4 hrs., at a temperature of 20 to 70 degrees Celsius.

7. Combination of a monosubstituted sulfamate derivate of the natural monosaccharide d-Fructose (Topiramate) with an anti-depressant from the phenyl ketone class (Bupropion) for treating obesity and plurimetabolic syndromes, in accordance with claims 1 or 4, wherein the dry granulation is conducted in 0.5 to 6 hrs., at a pressure of 0.5 to 10 Bar, at a temperature of 20 to 40 degrees Celsius.
